SEQUENCE LISTING (22 sequences)

SEQ ID NO: 1 - DNA, 6662 bp, Helianthus annuus
aagcttaatttataaagtttgagtttggtacgtgaatagtttttaaacatttactcaataagtgaaacttgggcctaggctcatttattatttataatttatttcattaatttatttataaacaattattgaagcttgggtctggactcgtttattaaacaaaccatttttagctcgacctcgtttaagtccgacttgatcgagcttttaatgactcaatcttgggtagctcacaagcgactcatatcatttagcccctaacttaatgctttggaacagaaaatgttagaccatgtgttatcgtctcacaaattttaaccaccagtcatacggtagctcctacatcactcacacatcatcagaccattttctttcatttttttcactgcacctcatagtaatgttactggttttaaccatccaccatccattaaaataatatttaaattatataagctaaaaatctttgatcggttgcttgccgacttggtattttttccatggtagcaaatgaaatattcctccaaggtgaaccggctaacgcggttctcccttttacagtcggtaacgccccttaagacagtccgatggccagtgatgtagccggggcgttaccttgtaacacatagcgttacaacaagcaacctttaaaacacttgtttgaaatcggtgaatgtttgtttgtttcttgtggcacctttaaaacacttgtcctttaaaacactatcggtgacaagtgttctctgaccaaggtcctttcgtccaatattcaaaacctcagggacactttatgaaaaatattctctctatatataaaccgctaaaccagcaacgctttctgtgaacggaagcacatcgtcgtctgtctcactagcggagactcaccgtttctacgaagtgaaaaccctaagcctctgtcgctcgatcgatcgatcgattcaggtattgtttgaatcatcgcatttcgataaatcattaattatcgaattttgcttttgatttttaatttaatttacttagctgctgcatttatgattgatgaacagatcagttgatttctcttctctgtgactttgatttggttcatttcactcagtttcctttaaatttgtgcgtttatctgtcttgcatgtgatcgattttgcttccttcatgtgttttcattcaatatgtactcttttaacttgtaaaatcttgcctgattgattttcttcttgatttttgtgatcaactctttaagtttttttttttttacaagattttctgttttagtcgagtcataaaagtgcgtaaatcattgacaggtgtcaattgtttatttttttttctggtagtaataataaaagcatcagcttttatattgggttgatgtattttcggtaaatgttttgcgttaaagctaaagaaacatgaatttcatggcttttgtaatcaagtgtaaattgacgactagatctacgaattatttctatttattttaaaacaaaatcgttaaatacaaaaataaatagtgagttcatttatcgttaaatacaaaaataaatagtgagttcattttatgcatgttttatcctagggtgaaaacggagacgagccgcgagttggagttcggtttatttataatttattaattagttattatgattattattattattattattattattattattattattattattattattttgtatataatttagtaatttttcataattttatatgtagtaattattattatttaaatatatattttatatatcaaattaaatattgtataaccgatgggcttgtttaggctcgtgagcgagctcgagttcaggctcgtttactaaacaagctcacctttagactcgggcttcaactcgtttaagcctggctcgttagagctttttttttctagctcggctcattttcacccttattttatcctttagaccagactcagattgttttttaacgttaaatccactcatatgccaataacatttgggcaaaaacgcattatgtctaaacacgttttttaaaacattattctttcaaaacatttttagataatataaaaccatgtgtagtaagtagtacacatgaataatgcccccatcaatgggcgttgtgcgacacgtgtcatcccagtcagcaaaggggcattatgggggtttttcatatatgggtgtgggacaatgccccatcaatgattacccaattattatcttttaaaacaaataatattacttggaagcttcttattggacaaataaataatgccccagcgtttagaccacacccccccccccctttctgaaaacaacgcccaaggggggcagtctggcggcgttaaggggcgtttttttggggaaaaacacccaaaaacccccagtacgggtggtctaagcataaaatgtgtttacaatatattttaaaaaacactgatttaatggagacaacatttaagacatactgtcatataatattattatacaaaagtagtaattagtaatcgtgttacgtaatataatgtgtatttgatagtctcgtatctactttttatgtatctatttcataaagaaaaggttaggcatgtttgtttgtaaatataaagatattgttcttgcttgcaggttgaaaagtctggtcaaacagtcaacatatgggtgcaggagaatacacgtctgtgaccaacgaaaacaacccactcgatcgagtccctcatgcaaaaccacccttcaccatcggcgatctgaaaaaagccatcccaccacactgcttccagcggtcgctaacccgttcgttctcctacgtgctgtctgacctcaccataaccgctgtcctctaccacattgccaccacctacttccaccacctccccacccctttgtcatccatcgcatgggcctcttactgggtagtccaaggctgcgtcctcaccggagtctgggtcatcgcccacgaatgtggtcaccatgcgtttagtgattatcaatgggtcgacgacactgtgggctttgttctccactcgtctttactcgtcccttacttttcgtggaaatatagtcaccaccgccaccattccaacactggatcgastcgagcgggacgaggttttcgtccccaaatcccgatcgaaagtcccgtggtactcgaaatactttaacaacacagtgggccgcattgtcagtatgttcgtcactctcactctcggctggcccttgtacttagctttcaatgtgtcgggccgaccctatgaccgtttcgcctgccactacgtcccaaccagccctatgtacaatgaacgtaaacgttaccagatagtcatgtccgacatcgggattgttatcacatcgttcatcctttatcgtgttgctatggcaaaagggttggtttgggtgatttgcgtctatggggttccgttgatggttgtgaacgcgtttctggtgttgatcacttatcttcaacatactcaccctggcttgccgcattatgatagctcggaatgggaatggttaaagggagcattggcgacagtggaccgtgactatggtgtgttgaacaaggtgttccatcatattaccgacacacatgtggtgcaccatttgttttcgacaatgcctcattataatgcgatggaagcacagaaggcgctgagaccggtgcttggggagtattatcggtttgacaagaccccgttttatgtagccatgtggagagagatgaaggaatgtttgtttgtggagcaagatgatgaagggaaaggaggtgtgttttggtacaagaataagatgaattaataagtgtttagtggatgtctatgcttattaagtctgtgacaatgggtcttgtcctggttctgggtctgggtctggttagtgtgtgtggttagtgtgtgttgtgggtcaagtgtatgtcagatggatgtaacgcagtatgtgttgccgtgttgggtgtgttttagaactattaatgaatatattatatatatctcttatttgggtgaactcwamtcrrgywatgsrtyyaamtgsgytrgsascwctcmcgactatkgtykayctkcasgcrkcyrckwrtymackagtgmytcaaaccaccacccactaacaacgttacatcaccaccgtagtcaccgccaccttcacttgtcgccactagcctccggccaccgtagtcaccgccatcttcacccatcgccaccagcctccgtaagatgagatatgaaggatgaacttaaattgattgtattttttcttaaacgatcatttcaaactccattttagcaagagcctatttgattcaaaccaaattgatattttttacacagctcgttttgacctgaactcttgcccagaacctgttttgacgtgaacctattttacctgccgattagcagtattgacggagccatgagtttcctttaggaattctcaaggttaaaagaaaaaagagattataaaaacaacaaaaatgaagattgaagaaacaacttgctacatatttgacagacgacataagcaaaattttactagcaaaacggtatatgaactcaaacgaacataacacatccttcgggttctacaaccaaagcaaaatttaactagcagacggtatattgtatatgaacacaaatgaacgtaggacctccttcgagttctacaaccaagatcacacatacatagtaattgtacaatgcaagggggacaagatcaaaccaatcgaaaagggaagatctgtggtagtctattaacaaatatacaaaccgggtccccataaacaaccataaaacattgaaactaccaaagcaagggtcatgggcgattagccacgtatgtaccttttgtgcgtcaagtaccataaccaatgacggaaaaccatgctagttataaggatatgaagcaaatcatgggatgtatgatcataatcagaatgtcaaaagatcactaatcttcctgagagattaatgcatacaattctttagcagtccaactttcttcaagaagctcactgagaggagccgcaatctcattcgccattgtcaacatgatgttcatgcaatctgtagccattcacagttcatcttaatcaacatgttcaaatttttaatatttttatgtaatataaaaggaaatgctaacctggaattttggtaaaattaaggagagatgtggcagcaagaatcttgtgctctacccatccactattttcgaggttttcttttagtttagagataagaaccgagaaagcagaaagctggaactcggactcaggtaaagaagcaagtgaagaactgaaccacaccatcgtgatcragyatactttcgtcatttcacgatgtccacaactcaagcactgggaaaatgaatccagaaacgagtttttcccatcactaacaagagacgttgacaatctgatcaaccaatcttcaacagcctcttcctcctcagtatcctataagccatagcatttggttttagagcgctttttatagattataaaattacaatttaaatccgtttaatttagctaatctttaataacatatttttttatagaacattaaaacgtttaactaagttacttttaaatctccaaaaagtaacaagaaaatatatatgtttttttctataatactaattgcgacagtaatgggctttagttatgttttatttgtaaaagaaatcccataatttatagtgtaaagtacacaaatggtccctatggttgaccaaaattttggatttggtctctagctttccaaaactacatgaatggtccctgtggtttgcactttgtaatgcatttagtccctagctaacaagtcttaaggttttcgcaggtctaggttattgagtaaggtgctgtttgtttttgcagacataaattgtctgcaagctgatttcatctgtttttatgtctgcaactgaagatgtggtctgaagatctgcaagctgaaaatataagactgtttgtttttataagttgataccggtctgaagcgcatcaaacaaagacgaaacattcacattcagcttcgatccagtggaggagaagagggaggtcggagaaggagctggtcaccggaggaggaaggaggtcgccggaggaggagggaggtcggagaagaggtaggtcgccggaggaggtaggtcaccggaggagaagagggaggtcgccggaggagaagagggatgtcggagaaggaggaggagcggtggtgggaggggctgatgtttgggagaggagaagagggaggtgtgaagatgttttaagaaaggggtccatagcttattttttaagatgaaaaaagctgagttcagatctgtttgaaaaaaaaaatcaaacagtcttcaagggtaacgtctgcgcgcttgcagacatcagcccccttgaagatgtttgaagaaaaaacaaacaccccctaaatgtgttacaaagtgcaaaccacagggaccatccatgtacttttgaaaagctagggaccaaatccaaaattttggttaagcatagggaccatccgtgtattttactctttgttataataggtagaggataatacaaaaaagaatactcatagtaaaacactaacactcattacattcgtacaaactcattacactgtccattccgttcctttctggtttcgttaaatataaataaagcaa

SEQ ID NO: 2 - DNA, 3397 bp, Helianthus annuus
cctttaaaacacttgtttgaaatcggtgaatgtttgtttgtttcttgtggcacctttaaaacacttgtcctttaaaacactatcggtgacaagtgttctctgaccaaggtcctttcgtccaatattcaaaacctcagggacactttatgaaaaatattctctctatatataaaccgctaaaccagcaacgctttctgtgaacggaagcacatcgtcgtctgtctcactagcggagactcaccgtttctacgaagtgaaaaccctaagcctctgtcgctcgatcgatcgatcgattcaggtattgtttgaatcatcgcatttcgataaatcattaattatcgaattttgcttttgatttttaatttaatttacttagctgctgcatttatgattgatgaacagatcagttgatttctcttctctgtgactttgatttggttcatttcactcagtttcctttaaatttgtgcgtttatctgtcttgcatgtgatcgattttgcttccttcatgtgttttcattcaatatgtactcttttaacttgtaaaatcttgcctgattgattttcttcttgatttttgtgatcaactctttaagtttttttttttttacaagattttctgttttagtcgagtcataaaagtgcgtaaatcattgacaggtgtcaattgtttatttttttttctggtagtaataataaaagcatcagcttttatattgggttgatgtattttcggtaaatgttttgcgttaaagctaaagaaacatgaatttcatggcttttgtaatcaagtgtaaattgacgactagatctacgaattatttctatttattttaaaacaaaatcgttaaatacaaaaataaatagtgagttcatttatcgttaaatacaaaaataaatagtgagttcattttatgcatgttttatcctagggtgaaaacggagacgagccgcgagttggagttcggtttatttataatttattaattagttattatgattattattattattattattattattattattattattattattattattttgtatataatttagtaatttttcataattttatatgtagtaattattattatttaaatatatattttatatatcaaattaaatattgtataaccgatgggcttgtttaggctcgtgagcgagctcgagttcaggctcgtttactaaacaagctcacctttagactcgggcttcaactcgtttaagcctggctcgttagagctttttttttctagctcggctcattttcacccttattttatcctttagaccagactcagattgttttttaacgttaaatccactcatatgccaataacatttgggcaaaaacgcattatgtctaaacacgttttttaaaacattattctttcaaaacatttttagataatataaaaccatgtgtagtaagtagtacacatgaataatgcccccatcaatgggcgttgtgcgacacgtgtcatcccagtcagcaaaggggcattatgggggtttttcatatatgggtgtgggacaatgccccatcaatgattacccaattattatcttttaaaacaaataatattacttggaagcttcttattggacaaataaataatgccccagcgtttagaccacacccccccccccctttctgaaaacaacgcccaaggggggcagtctggcggcgttaaggggcgtttttttggggaaaaacacccaaaaacccccagtacgggtggtctaagcataaaatgtgtttacaatatattttaaaaaacactgatttaatggagacaacatttaagacatactgtcatataatattattatacaaaagtagtaattagtaatcgtgttacgtaatataatgtgtatttgatagtctcgtatctactttttatgtatctatttcataaagaaaaggttaggcatgtttgtttgtaaatataaagatattgttcttgcttgcaggttgaaaagtctggtcaaacagtcaacatatgggtgcaggagaatacacgtctgtgaccaacgaaaacaacccactcgatcgagtccctcatgcaaaaccacccttcaccatcggcgatctgaaaaaagccatcccaccacactgcttccagcggtcgctaacccgttcgttctcctacgtgctgtctgacctcaccataaccgctgtcctctaccacattgccaccacctacttccaccacctccccacccctttgtcatccatcgcatgggcctcttactgggtagtccaaggctgcgtcctcaccggagtctgggtcatcgcccacgaatgtggtcaccatgcgtttagtgattatcaatgggtcgacgacactgtgggctttgttctccactcgtctttactcgtcccttacttttcgtggaaatatagtcaccaccgccaccattccaacactggatcactcgagcgggacgaggttttcgtccccaaatcccgatcgaaagtcccgtggtactcgaaatactttaacaacacagtgggccgcattgtcagtatgttcgtcactctcactctcggctggcccttgtacttagctttcaatgtgtcgggccgaccctatgaccgtttcgcctgccactacgtcccaaccagccctatgtacaatgaacgtaaacgttaccagatagtcatgtccgacatcgggattgttatcacatcgttcatcctttatcgtgttgctatggcaaaagggttggtttgggtgatttgcgtctatggggttccgttgatggttgtgaacgcgtttctggtgttgatcacttatcttcaacatactcaccctggcttgccgcattatgatagctcggaatgggaatggttaaagggagcattggcgacagtggaccgtgactatggtgtgttgaacaaggtgttccatcatattaccgacacacatgtggtgcaccatttgttttcgacaatgcctcattataatgcgatggaagcacagaaggcgctgagaccggtgcttggggagtattatcggtttgacaagaccccgttttatgtagccatgtggagagagatgaaggaatgtttgtttgtggagcaagatgatgaagggaaaggaggtgtgttttggtacaagaataagatgaattaataagtgtttagtggatgtctatgcttattaagtctgtgacaatgggtcttgtcctggttctgggtctgggtctggttagtgtgtgtggttagtgtgtgttgtgggtcaagtgtatgtcagatggatgtaacgcagtatgtgttgccgtgttgggtgtgttttagaactattaatgaatatattatatatatctcttatttgggtgaactcaaatcgggtaatgggtttaaatgggctagcaccactca

SEQ ID NO: 3 - DNA, 872 bp, Helianthus annuus
gagaagagggaggtgtgaagatgttttaagaaaggggtccatagcttattttttaagatgaaaaaagctgagttcagatctgtttgaaaaaaaaaatcaaacagtcttcaagggtaacgtctgcgcgcttgcagacatcagcccccttgaagatgtttgaagaaaaaacaaacaccccctaaatgtgttacaaagtgcaaaccacagggaccatccatgtacttttgaaaagctagggaccaaatccaaaattttggttaagcatagggaccatccgtgtattttactctttgttataataggtagaggataatacaaaaaagaatactcatagtaaaacactaacactcattacattcgtacaaactcattacactgtccattccgttcctttctggtttcgttaaatataaataaagcaacagtacgggtggtctaagcataaaatgtgtttacaatatattttaaaaaacactgatttaatggagacaacatttaagacatactgtcatataatattattatacaaaagtagtaattagtaatcgtgttacgtaatataatgtgtatttgatagtctcgtatctactttttatgtatctatttcataaagaaaaggttaggcatgtttgtttgtaaatataaagatattgttcttgcttgcaggttgaaaagtctggtcaaacagtcaacatatgggtgcaggagaatacacgtctgtgaccaacgaaaacaacccactcgatcgagtccctcatgcaaaaccacccttcaccatcggcgatctgaaaaaagccatcccaccacactgcttccagcggtcgctaacccgttcgttctcctacgtgctgtctgacctcaccataaccgct

SEQ ID NO: 4 - DNA, 3026 bp, Helianthus annuus
caaaccaccacccactaacaacgttacatcaccaccgtagtcaccgccaccttcacttgtcgccactagcctccggccaccgtagtcaccgccatcttcacccatcgccaccagcctccgtaagatgagatatgaaggatgaacttaaattgattgtattttttcttaaacgatcatttcaaactccattttagcaagagcctatttgattcaaaccaaattgatattttttacacagctcgttttgacctgaactcttgcccagaacctgttttgacgtgaacctattttacctgccgattagcagtattgacggagccatgagtttcctttaggaattctcaaggttaaaagaaaaaagagattataaaaacaacaaaaatgaagattgaagaaacaacttgctacatatttgacagacgacataagcaaaattttactagcaaaacggtatatgaactcaaacgaacataacacatccttcgggttctacaaccaaagcaaaatttaactagcagacggtatattgtatatgaacacaaatgaacgtaggacctccttcgagttctacaaccaagatcacacatacatagtaattgtacaatgcaagggggacaagatcaaaccaatcgaaaagggaagatctgtggtagtctattaacaaatatacaaaccgggtccccataaacaaccataaaacattgaaactaccaaagcaagggtcatgggcgattagccacgtatgtaccttttgtgcgtcaagtaccataaccaatgacggaaaaccatgctagttataaggatatgaagcaaatcatgggatgtatgatcataatcagaatgtcaaaagatcactaatcttcctgagagattaatgcatacaattctttagcagtccaactttcttcaagaagctcactgagaggagccgcaatctcattcgccattgtcaacatgatgttcatgcaatctgtagccattcacagttcatcttaatcaacatgttcaaatttttaatatttttatgtaatataaaaggaaatgctaacctggaattttggtaaaattaaggagagatgtggcagcaagaatcttgtgctctacccatccactattttcgaggttttcttttagtttagagataagaaccgagaaagcagaaagctggaactcggactcaggtaaagaagcaagtgaagaactgaaccacaccatcgtgatcaagcatactttcgtcatttcacgatgtccacaactcaagcactgggaaaatgaatccagaaacgagtttttcccatcactaacaagagacgttgacaatctgatcaaccaatcttcaacagcctcttcctcctcagtatcctataagccatagcatttggttttagagcgctttttatagattataaaattacaatttaaatccgtttaatttagctaatctttaataacatatttttttatagaacattaaaacgtttaactaagttacttttaaatctccaaaaagtaacaagaaaatatatatgtttttttctataatactaattgcgacagtaatgggctttagttatgttttatttgtaaaagaaatcccataatttatagtgtaaagtacacaaatggtccctatggttgaccaaaattttggatttggtctctagctttccaaaactacatgaatggtccctgtggtttgcactttgtaatgcatttagtccctagctaacaagtcttaaggttttcgcaggtctaggttattgagtaaggtgctgtttgtttttgcagacataaattgtctgcaagctgatttcatctgtttttatgtctgcaactgaagatgtggtctgaagatctgcaagctgaaaatataagactgtttgtttttataagttgataccggtctgaagcgcatcaaacaaagacgaaacattcacattcagcttcgatccagtggaggagaagagggaggtcggagaaggagctggtcaccggaggaggaaggaggtcgccggaggaggagggaggtcggagaagaggtaggtcgccggaggaggtaggtcaccggaggagaagagggaggtcgccggaggagaagagggatgtcggagaaggaggaggagcggtggtgggaggggctgatgtttgggagaggagaagagggaggtgtgaagatgttttaagaaaggggtccatagcttattttttaagatgaaaaaagctgagttcagatctgtttgaaaaaaaaaatcaaacagtcttcaagggtaacgtctgcgcgcttgcagacatcagcccccttgaagatgtttgaagaaaaaacaaacaccccctaaatgtgttacaaagtgcaaaccacagggaccatccatgtacttttgaaaagctagggaccaaatccaaaattttggttaagcatagggaccatccgtgtattttactctttgttataataggtagaggataatacaaaaaagaatactcatagtaaaacactaacactcattacattcgtacaaactcattacactgtccattccgttcctttctggtttcgttaaatataaataaagcaacagtacgggtggtctaagcataaaatgtgtttacaatatattttaaaaaacactgatttaatggagacaacatttaagacatactgtcatataatattattatacaaaagtagtaattagtaatcgtgttacgtaatataatgtgtatttgatagtctcgtatctactttttatgtatctatttcataaagaaaaggttaggcatgtttgtttgtaaatataaagatattgttcttgcttgcaggttgaaaagtctggtcaaacagtcaacatatgggtgcaggagaatacacgtctgtgaccaacgaaaacaacccactcgatcgagtccctcatgcaaaaccacccttcaccatcggcgatctgaaaaaagccatcccaccacactgcttccagcggtcgctaacccgttcgttctcctacgtgctgtctgacctcaccataaccgct

SEQ ID NO: 5 - DNA, 19 bp, Artificial Sequence
  Description of Artificial Sequence Synthetic oligonucleotide
caaaccaccacccactaac

SEQ ID NO: 6 - DNA, 20 bp, Artificial Sequence
  Description of Artificial Sequence Synthetic oligonucleotide
gagaagagggaggtgtgaag

SEQ ID NO: 7 - DNA, 20 bp, Artificial Sequence
  Description of Artificial Sequence Synthetic oligonucleotide
agcggttatggtgaggtcag

SEQ ID NO: 8 - DNA, 20 bp, Artificial Sequence
  Description of Artificial Sequence Synthetic oligonucleotide
acaaagcccacagtgtcgtc

SEQ ID NO: 9 - DNA, 20 bp, Artificial Sequence
  Description of Artificial Sequence Synthetic oligonucleotide
gccatagcaacacgataaag

SEQ ID NO: 10 - DNA, 45 bp, Artificial Sequence
  Description of Artificial Sequence Synthetic oligonucleotide
ttattattattattattattattattattattattattattatta

SEQ ID NO: 11 - DNA, 20 bp, Artificial Sequence
  Description of Artificial Sequence Synthetic oligonucleotide
ttggagttcggtttatttat

SEQ ID NO: 12 - DNA, 19 bp, Artificial Sequence
  Description of Artificial Sequence Synthetic oligonucleotide
ttagtaaacgagcctgaac

SEQ ID NO: 13 - DNA, 763 bp, Helianthus annuus
  modified_base (696)..(696): a, c, t, g, unknown or other
cactaacactcattacattcgtacaaactcattacactgtccattccgttcctttctggtttcgttaaatataaataaagcaacagtacgggtggtctaagcataaaatgtgtttacaatatattttaaaaaacactgatttaatggagacaacatttaagacatactgtcatataatattattatacaaaagtagtaattagtaatcgtgttacgtaatataatgtaccagcccgggccgtcgaccacgcgtgccctatagtcactaacactcattacattcgtacaaactcattacactgtccattccgttcctttctggtttcgttaaatataaataaagcaaaaaacaccgtgattccatgaactaaaaacacaagaccccaatcctgcaaccataacagcgtaaaaaaatctacagagctttaggaagagagaaaattaccgtcaaaatgttggtggcgttgtactctaggacatctaattcaggctgaataataacatccaattcaggtctattatagaaaccagctttctgtagattataatcctgtgtgattaccgtcccagataaagagatacgcgctcccattataagaagtgctctacaacatgtttctcgcaccttttcatcaaaaagactagtgtcaagagcagatgtaattgtatcaacagcttcctctctatatatactgnacttctgttgatccacctgtcagttggaatacaaacatcttaacaatcaaatatgtgtgacaatttt

SEQ ID NO: 14 - DNA, 21 bp, Artificial Sequence
  Description of Artificial Sequence Synthetic oligonucleotide
ttttactctttgttataatag

SEQ ID NO: 15 - DNA, 20 bp, Artificial Sequence
  Description of Artificial Sequence Synthetic oligonucleotide
tttttagttcatggaatcac

SEQ ID NO: 16 - DNA, 22 bp, Artificial Sequence
  Description of Artificial Sequence Synthetic oligonucleotide
acactaacactcattacattcg

SEQ ID NO: 17 - DNA, 20 bp, Artificial Sequence
  Description of Artificial Sequence Synthetic oligonucleotide
cctaaagctctgtagatttt

SEQ ID NO: 18 - DNA, 22 bp, Artificial Sequence
  Description of Artificial Sequence Synthetic oligonucleotide
aaagcaaaaaacaccgtgattc

SEQ ID NO: 19 - DNA, 21 bp, Artificial Sequence
  Description of Artificial Sequence Synthetic oligonucleotide
ggtgttattattcagcctgaa

SEQ ID NO: 20 - DNA, 21 bp, Artificial Sequence
  Description of Artificial Sequence Synthetic oligonucleotide
gatgttattattcagcctgaa

SEQ ID NO: 21 - DNA, 51 bp, Artificial Sequence
  Description of Artificial Sequence Synthetic oligonucleotide
ttattattattattattattattattattattattattattattattatta

SEQ ID NO: 22 - DNA, 48 bp, Artificial Sequence
  Description of Artificial Sequence Synthetic oligonucleotide
ttattattattattattattattattattattattattattattatta

(19) United States
(12) Patent Application Publication
(10) Pub. No.: US 2009/0258346 A1
(43) Pub. Date: Oct. 15, 2009

(54) Method of selecting sunflower genotypes with high oleic acid content in seed oil

(76) Inventors: Andre Berville, Montpellier (FR); Severine Lacombe, Luc (FR); Stanislas Veillet, Anglet (FR); Christel Granier, Bayonne (FR); Sandrine Leger, Anglet (FR); Philippe Jouve, Montech (FR)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/587,956

(22) PCT Filed: Apr. 27, 2005
(86) PCT No.: PCT/EP2005/004635

§ 371 Date: Sep. 5, 2007

(30) Foreign Application Priority Data
Apr. 29, 2004 (EP) 04291102.4

(51) Int. Cl. C12Q 1/68; C07H 21/00

(52) U.S. Cl. 435/6; 536/23.1

(57) ABSTRACT

The present invention relates to the selection of sunflower genotypes with high oleic acid content in seed oil. The invention concerns more particularly molecular markers useful for a rapid and easy selection of sunflower lines and then sunflower hybrids capable of producing seeds having high oleic acid content.

The present invention relates to the selection of sunflower genotypes with high oleic acid content in seed oil. The invention concerns more particularly molecular markers useful for a rapid and easy selection of sunflower lines and then sunflower hybrids capable of producing seeds having high oleic acid content.

In the present description, the expression “high oleic acid content seeds” designates seeds containing more than 60% of oleic acid.

### BACKGROUND OF THE INVENTION

Since the 1960s, vegetable oils rich in unsaturated fatty acids have become particularly important due to the relationship established between the saturated fatty acids present notably in animal fats and the cholesterol level increase.

Most of the fatty acids in vegetable oils are fatty acids having 16 or 18 carbon atoms. The C16-fatty are generally saturated fatty acids (16:0=palmitic acid). Conversely, the C18 fatty acids are either saturated (18:0=stearic acid) or unsaturated having 1, 2 or 3 double bonds (18:1=oleic acid; 18:2=linoleic acid; 18:3=linolenic acid).

Certain unsaturated fatty acids, notably linolenic acid, are not synthesized by the human body and are of an insufficient amount in fats of animal origin. Among the unsaturated fatty acids, the mono-unsaturated 18:1 oleic acid, has properties which is particularly suitable for preventing cardiovascular diseases. Moreover, High oleic acid content oils are more resistant to heating and are thus recommended for frying.

The fatty acids composition in vegetable oils deriving from seeds is variable depending on the oleaginous plant. Olive oil has naturally high 18:1 content (around 70%), nevertheless, its non-lipid part of the oil decreases the interest of this oil. Normal sunflower (hereinafter named LO varieties) oil contains mainly linoleic acid and high levels of phytosterols and tocopherol conferring hypocholesterolemic and anti-oxidant properties to the non-lipid part of the oil. Sunflower lines and hybrids, which have high 18:1 content in their seeds (hereinafter named HO varieties) have been obtained by selection programs from the HO Pervenets mutant, said mutant being obtained by chemical mutagenesis (1). They are particularly interesting since they have the combined benefits of oleic acid and the non-lipid part of the sunflower oil (phytosterol and tocopherol). They therefore respond to the requirement of quality by consumers.

In addition to their dietary benefits, vegetable oils are of significant industrial interest. Indeed, they represent a source of fatty acids as starting material used in the lipochemical industry (detergents, paints, cosmetics). Vegetable oil from the HO sunflower type is a substantially pure industrial source of 18:1, that reduces the purification steps.

In order to respond to the needs of vegetable oils of food or industrial interest, the improvement of the oleaginous varieties is concerned with the control and the change of their fatty acid compositions by means of conventional selection programs, but also by mutagenesis and transgenesis.

The chemical mutagenesis carried out by Soldatov in 1976 (1) on a sunflower population allowed the Pervenets mutant population to be obtained. The average content of 18:1 of the seeds from this population is higher than 65%, the individual contents being between 60 and 80% whereas in normal LO varieties this content is about 20%. The Pervenets population was distributed throughout the world and used in many selection programs in order to convert selected genotypes with low 18:1 content into genotypes having 18:1 content in their seeds higher than 80%.

During the conversion process of the normal LO material into HO one, the selection is generally based on the 18:1 content of the seeds. This phenotypic determination does not allow a rapid and early detection of HO genotypes and cannot distinguish between homozygote and heterozygote genotypes for the mutation. It is therefore necessary to have selection markers at genomic level allowing earlier and rapid determination of the HO phenotype homozygous for the mutation during conversion process.

The accumulation of the 18:1 in the seeds is mainly dependent on two enzymatic reactions: the upstream desaturation of 18:0 into 18:1 and the downstream desaturation of 18:1 into 18:2. Studies carried out by Garcés and Mancha in 1989 and 1991 (2, 3) demonstrated that the HO phenotype is associated with a marked activity decrease of the Δ12-desaturase enzyme, which catalyses the desaturation of 18:1 into 18:2 in the HO seeds during the critical stages of constructing the lipidic stocks, explaining the 18:1 accumulation. Kabbaj et al (4) subsequently demonstrated that one HO genotype presents a significant decrease in the Δ12-desaturase mRNA levels in the seeds during the critical stages of producing the lipidic stocks compared to 2 LO genotypes. This decrease explains the decrease of the amount of enzyme and therefore of Δ12-desaturase activity already demonstrated. Hongtrakul et al. (5) and then Lacombe et al. (6, 7) showed that HO lines derived from Pervenets mutant carry specific restriction fragment length polymorphisms (RFLPs) revealed using a Δ12-desaturase cDNA as a probe. These RFLPs determine the Δ12 HO specific allele, Δ12HOS. The normal LO lines do not carry the Δ12HOS allele but another allele named Δ12LOR at this locus (named Δ12HL locus) (6, 7). Studying the inheritance of the phenotype HO in an F2 population, Lacombe et al. (6) revealed that the HO phenotype co-segregated with the Δ12HOS allele pointing out that the Pervenets mutation is carried or genetically tightly linked to the Δ12HOS allele. In another segregating population (recombinant inbred lines F6 population), Lacombe et al. (2) showed that all the HO lines carry the Δ12HOS allele. However, the recombinant lines carrying Δ12HOS are equally shared into HO or LO classes. The absence of HO plants carrying the Δ12LOR allele eliminates the occurrence of a recombination event between the Δ12HL locus and the locus containing the Pervenets mutation. In the genetic background of this population, the HO phenotype is therefore due to two independent loci: the Δ12HL locus carrying the Δ12HOS allele and consequently the Pervenets mutation, and another locus, where an allele suppresses the effect of the Δ12HOS allele leading to restore the LO phenotype.

Once the coincidence or the very tightly genetic linkage between the Pervenets mutation and the Δ12HOS allele revealed, Lacombe et al. (8) established physical maps of the Δ12HOS and Δ12LOR alleles at the Δ12HL locus (FIG. 1). For this purpose, they used the HO and LO RFLP profiles revealed with the Δ12-desaturase cDNA used as a probe. The Δ12LOR allele in LO genotypes corresponds to a 5.8 kb EcoRI and to a 8 kb HindIII fragments carrying Δ12-desaturase like sequences. The double digest with EcoRI+HindIII leads to a 2.2 kb fragment. For the Δ12HOS allele in HO genotypes, the 5.8 kb EcoRI fragment is still present but another 7.9 kb EcoRI extra fragment is also revealed. With HindIII, the 8 kb fragment revealed in LO genotypes lengthens to 16 kb in HO genotypes. With the EcoRI+HindIII double digest, the Δ12HOS allele displayed the 2.2 kb fragment revealed in Δ12LOR allele plus the 7.9 kb EcoRI extra fragment. According to all these data, the Δ12HOS and Δ12LOR allele physical maps were established. The Δ12LOR allele carries one region whereas the Δ12HOS allele carries 2 adjacent regions with Δ12-desaturase like sequences: the Δ12HOS/Δ12LOR common region determined by the common 5.8 kb EcoRI fragment and a HO specific region determined by the 7.9 kb EcoRI extra fragment (FIG. 1, ref 8).

Studying genomic clones carrying Δ12-desaturase like sequence from a HO genotype genomic library, Lacombe et al. (8) revealed that the Δ12HOS/Δ12LOR common fragment should carry a functional Δ12-desaturase gene interrupted by a 1686 bp intron in the 5′UTR part of the gene.

### SUMMARY OF THE INVENTION

The present invention relates to molecular markers that are strictly linked to genetic factors involved in 18:1 accumulation in seed oil of HO genotypes of sunflower: the Pervenets mutation and another independent factor, the supole factor.

The invention relates to PCR molecular markers that partly amplify the Pervenets mutation. It also relates to a SSR marker (15-17 TTA motifs) in the intron of the Δ12-desaturase functional gene adjacent to the mutation that enables to map the gene locus in most of segregating populations and to select genotypes carrying the Pervenets mutation.

The invention also relates to specific markers of a genetic factor suppressing the Pervenets mutation effect. The presence of the suppressor (supole) is revealed in genotypes by combining the presence of the Δ12HOS allele and the low 18:1 content in seed oil.

All these molecular markers may represent advantageous and useful tools in selection programs for rapid screening and early detection of HO genotypes producing seeds with a high 18:1 content, carrying the Pervenets mutation without the unfavourable supole factor by means of the PCR technology.

The invention also relates to processes for the selection of sunflower producing seeds with a high oleic acid content.

The present invention also relates to primers useful for nucleic acid amplification and to combinations thereof.

Finally, the invention concerns the test kits for selecting sunflower having seeds with a high content of oleic acid which contain at least one of the above combinations.

### DETAILED DESCRIPTION OF THE INVENTION

The present invention relates to Pervenets mutation specific molecular markers located in a 29 kb region, a map thereof is represented on FIG. 2. This region carrying these molecular markers is divided in 3 sequences, from 5′ to 3′:
sequence N1 having 6.662 kb: Δ12HOS/Δ12LOR common part carrying a Δ12-desaturase gene (SEQ ID NO:1);
sequence N2: HO specific insertion; (this sequence has been partially sequenced (SEQ ID NO:2);
sequence N3: the 3′ adjacent part of the HO specific insertion fragment.

The molecular marker of the invention comprises the isolated nucleic acid SEQ ID No 3 having 872 bp or a sequence having a high degree of homology with sequence SEQ ID No 3. Said nucleic acid sequence is a part of sequence SEQ ID NO:4 having 3026 bp and being identified as sequence N1/2 on FIG. 2. The marker having SEQ ID NO:3 is located between 2154 bp and 3026 bp of SEQ ID NO:4.

In the present description, a high degree of homology denotes a homology (ratio of the identical nucleotides to the total number of nucleotides) of at least 85%, and preferably 90%, for the nucleotide sequences when they are aligned according to the maximum homology by the optimal sequence alignment method of ALIGN. This method is used especially in the GCG software of Devereux et al. (9).

The invention also relates to the isolated nucleic acid fragments having 10-30 bp and which hybridize with SEQ N2 (SEQ ID NO:2). Said fragments are particularly useful tools as primers for nucleic acid amplification, for example PCR amplification.

Examples of said fragments are the fragments having the following sequences:

(SEQ ID N^{o}7)

sequence N2-1R: 5′-AGCGGTTATGGTGAGGTCAG-3′

(SEQ ID N^{o}8)

sequence N2-2R: 5′-ACAAAGCCCACAGTGTCGTC-3′

(SEQ ID N^{o}9)

sequence N2-3R: 5′-GCCATAGCAACACGATAAAG-3′

The invention also relates to the primer pairs useful for nucleic acid amplification, which comprise:

1) as the first member of the pair a nucleic acid fragment having 1 to 30 bp and which hybridises with sequence N1 (SEQ ID No 1) and

2) as the second member of the pair a nucleic acid fragment having 10 to 30 bp and which hybridises with SEQ ID NO:2.

Examples of such pairs of primers comprise as:

1) first member:

(SEQ ID N^{o}5)

sequence N1-2F: 5′-CAAACCAACCACCCACTAAC-3′
or

(SEQ ID N^{o}6)

sequence N1-3F: 5′-GAGAAGAGGGAGGTGTGAAG-3′
2) second member:

(SEQ ID N^{o}7)

sequence N2-1R: 5′-AGCGGTTATGGTGAGGTCAG-3′

(SEQ ID N^{o}8)

sequence N2-2R: 5′-ACAAAGCCCACAGTGTCGTC-3′

(SEQ ID N^{o}9)

sequence N2-3R: 5′-GCCATAGCAACACGATAAAG-3′

In the present description, acid nucleic sequences codes end by a number whereas acid nucleic fragments useful as primers end by F or R depending on they are designed forward or reverse, respectively. These references are reported on the map of the 29 kb region (FIG. 2).

The invention also relates to the isolated SSR nucleic acid sequence SEQ ID No 10 having 45 bp with 16 TTA repeat microsallelites TTA. This sequence involving a polymorphism associated with the Pervenets mutation may be used in selection programs for rapidly identifying the locus of the Pervenets mutation.

Said SSR sequence is located in the intron of the oleate-desaturase gene besides the Pervenets insertion.

Therefore, said SSR sequence is very useful for mapping the oleate-desaturase gene in the genome of sunflower plants to be selected.

The invention also concerns the molecular marker specific for mapping the oleate-desaturase gene in the genome of the sunflower to be selected which comprises the nucleic acid sequence SEQ ID No 10 or a sequence having a high degree of homology with sequence SEQ ID No 10.

The invention also concerns the isolated nucleic acid fragments SEQ ID No 11 and SEQ ID No 12. Said fragments are useful as primers for amplifying the above SSR sequence:

(SEQ ID N^{o}11)

sequence N1-1F: 5′-TTGGAGTTCGGTTTATTTAT-3′

(SEQ ID N^{o}12)

sequence N1-1R: 5′-TTAGTAAACGAGCCTGAAC-3′

All the isolated nucleic acid sequences and fragments of the invention may be obtained by chemical synthesis following the conventional methods well known by the person skilled in the art.

The invention also relates to a process for selecting sunflower having seeds with a high content of oleic acid which comprises the steps of:
extracting the genomic DNA;
amplifying said genomic DNA by means of primer pair, said pair being constituted with a first member of nucleic acid fragment having 10 to 30 bp, which hybridises with sequence N1 (SEQ ID NO:1) and a second member of nucleic acid fragment having 10 to 30 bp, which hybridises with sequence N2 (SEQ ID NO:2);
hybridizing the amplified DNA fragment with the labelled sequence SEQ ID No 3 and
isolating the genotypes giving a positive hybridisation signal.

This process allows the selection of genotypes having the Pervenets mutation without needing to determine the oleic acid content of the seeds, which considerably reduces the selection time.

Following an advantageous embodiment of this process, the Pervenets mutation is firstly mapped in most of progenies (most of sunflower line couples display polymorphism for the SSR) by a process comprising the steps of:
extracting the genomic DNA;
amplifying said genomic DNA by means of primers SEQ ID No 11 and SEQ ID No 12;
isolating the clones having a DNA fragment length which is different from a reference clone without the Pervenets mutation.

The thus selected clones have the Pervenets mutation and are used as such in the above invention process.

Examples of the primer pairs useful in the invention process are the:
isolated nucleic acid fragments SEQ ID NO:5 and SEQ ID NO:6 as first member of said pair, and
isolated nucleic acid fragments SEQ ID NO:7 to SEQ ID NO:9 as second member of said pair.

The invention also relates to the molecular marker constituted by the isolated nucleic sequence SEQ ID NO:13 having 763 bp, which is common to LO and HO regions and located over the insertion point of the Pervenets mutation. The insertion point is located between the bp 83 and bp 366 of SEQ ID NO:13.

The invention also relates to the isolated nucleic acid fragments having 10-30 bp and which hybridise with SEQ ID NO:13. Said fragments are also useful tools as primers for nucleic acid amplification, for example for PCR amplification.

Examples of said fragments are the fragments having the following sequences:

(SEQ ID N^{o}14)

| | |
|---|---|
| sequence N2-1F: | 5′-TTTTACTCTTTGTTATAATAG-3′ |

(SEQ ID N^{o}16)

| | |
|---|---|
| sequence N2-2F: | 5′-ACACTAACACTCATTACATTCG-3′ |

(SEQ ID N^{o}18)

| | |
|---|---|
| sequence N2-3F: | 5′-AAAGCAAAAAACACCGTGATTC-3′ |

(SEQ ID N^{o}15)

| | |
|---|---|
| sequence N3-1R: | 5′-TTTTTAGTTCATGGAATCAC-3′ |

(SEQ ID N^{o}17)

| | |
|---|---|
| sequence N3-2R: | 5′-CCTAAAGCTCTGTAGATTTT-3′ |

(SEQ ID N^{o}19)

| | |
|---|---|
| sequence N3-3R: | 5′-GGTGTTATTATTCAGCCTGAA-3′ |

(SEQ ID N^{o}20)

| | |
|---|---|
| sequence N3-3′R: | 5′-GATGTTATTATTCAGCCTGAA-3′ |

The invention also relates to the process for amplifying the region across the insertion Pervenets mutation site both in LO and HO genotypes. Said process comprises the steps of:
extracting the genomic DNA;
amplifying said genomic DNA by means of primer pair, said pair being constituted with a first member of nucleic acid fragments having 10 to 30 bp which hybridises with sequence N1 (SEQ ID NO:13).

In all the invention processes, the amplification step may be carried out by any nucleic amplification method known by the person skilled in the art, for example by the well-known PCR amplification method.

A 6662 kb region carrying the Δ12HOS and Δ12LOR common part in RHA 345 HO line (SEQ N1) was cloned, sequenced and characterized. It was shown that it carries a putative functional Δ12-desaturase gene (SEQ N1-Δ12). Indeed, the TATAAA and CAAT consensus promoter elements are present at positions −92 bp and −42 bp upstream the +1 transcriptional point, respectively. Because of its sequence and its position, the AAGTAA sequence, 16 nt before the end of the transcribed part, corresponds to a poly-A signal AATAAA, except for one nucleotide. The Δ12-desaturase gene is interrupted by a 1686pb intron between nt 83 and nt 1767 upstream the +1 transcriptional point. The consensus splicing sites GT and AG are present at the intron extremities. A 16 repeats of a TTA SSR motive is revealed in the intron sequence between nt 784 and 832 upstream the +1 transcriptional point. Using primer pairs enabling this SSR amplification (SEQ N1-1F and SEQ N1-1R), size polymorphism at this locus in a set of 42 HO and LO genotypes was tested (Table 1).

Sequence N1-1F = 5′-TTGGAGTTCGGTTTATTTAT-3′
Sequence N1-1R = 5′-TTAGTAAACGAGCCTGAAC-3′

PCR amplification leads to 237/240/243 bp fragments corresponding to 15/16/17 SSR repeats, respectively. In the 174 individuals segregating population already studied (7), a strict co-segregation between the SSR polymorphism of the HO parental line (16 motifs) and the Δ12HOS allele (EXAMPLE 2) was revealed. So, this SSR sequence displays a polymorphism tightly linked to the Pervenets mutation. Consequently, it can be used to map the locus of the Δ12-desaturase gene in sunflower genome in most of crosses. Moreover, it may be used in selection programs for fast screening of genotypes carrying the Pervenets mutation by PCR method.

The invention relates to this SSR nucleic acid sequence present in SEQ ID N1 and SEQ ID N1-Δ12. The invention also relates to sequences having a high degree of homology with sequence SEQ ID N1 or N1-Δ12 and nucleic acid fragments that can be used as PCR primers to amplify the SSR motive such as SEQ ID N1-1F and SEQ ID N1-1R leading to a PCR fragment carrying the 15, 16 or 17 TTA motives.

In order to select molecular markers corresponding to the Pervenets mutation itself, parts of the 7.9 kb EcoRI HO specific fragment that should carry the Pervenets mutation was cloned, sequenced and characterized. Primer pairs on both side of the 5′ Pervenets mutation insertion point: SEQ ID N1-2F located on SEQ N1 and SEQ ID N2-1R located on the SEQ N2 and designed in Δ12-desaturase cDNA were selected.

Sequence N1-2F = 5′-CAAACCAACCACCCACTAAC-3′
Sequence N2-1R = 5′-AGCGGTTATGGTGAGGTCAG-3′

Because of their positions, these primers lead to a 3026 bp PCR amplification fragment only in RHA 345 HO genotype compared to LO4 and other LO genotypes. Subsequently, we cloned sequenced and characterised this PCR fragments carrying the 5′ Pervenets mutation insertion point (SEQ ID N1/2) was cloned, sequenced and characterized. The organisation of this sequence is, from 5′ to 3′:
A 2576 bp HO/LO common region
the 5′ insertion point
HO specific Δ12-desaturase gene like sequence: part of the intron (239 bp) and part of the coding region (211 bp).

Based on this characterisation, new primers were computed (SEQ ID N1-3F located on SEQ ID N1, SEQ ID N2-2R and SEQ ID N2-3R designed on Δ12-desaturase cDNA sequence).

Sequence N1-3F = 5′-GAGAAGAGGGAGGTGTGAAG-3′
Sequence N2-2R = 5′-ACAAAGCCCACAGTGTCGTC-3′
Sequence N2-3R = 5′-GCCATAGCAACACGATAAAG-3′

These primer pair combinations lead to HO specific amplification fragments of about 870 bp (SEQ ID N1-3F+SEQ ID N2-1R), 1000 bp (SEQ ID N1-3F+SEQ ID N2-2R) and 1400 bp (SEQ ID N1-3F+SEQ ID N2-3R) in PCR experiments involving 42 HO and LO genotypes (EXAMPLE 1). In the 174 recombinant inbred line population, a strict co-segregation between the SEQ ID N1-3F+N2-1R HO specific fragment and the Δ12HOS allele (EXAMPLE 2) was revealed.

The invention relates to SEQ N1/2 (SEQ ID NO:1) corresponding to part of the Δ12-desaturase HO specific fragment, and sequences having a high degree of homology with SEQ N1/2 (SEQ ID NO:1). The invention also relates to nucleic acid fragments which can be used as PCR primers to amplify genomic region having a high degree of homology with SEQ ID N1/2 such as combinations of SEQ ID N1-2F or SEQ ID N1-3F with SEQ ID N2-1R, SEQ ID N2-2R and SEQ ID N2-3R. These primer pair combinations enable to obtain fragment lengths of 3026 bp (sequence N1-2F+sequence N2-1R), 870 bp (sequence N1-3F+sequence N2-1R), 1000 bp (sequence N1-3F+sequence N2-2R) and 1400 bp (sequence N1-3F+sequence N2-3R), respectively. Those primer sequences are coded:

Sequence-N1-2F = 5′-CAAACCACCACCCACTAAC-3′
Sequence-N1-3F = 5′-GAGAAGAGGGAGGTGTGAAG-3′
Sequence-N2-1R = 5′-AGCGGTTATGGTGAGGTCAG-3′
Sequence-N2-2R = 5′-ACAAAGCCCACAGTGTCGTC-3′
Sequence-N2-3R = 5′-GCCATAGCAACACGATAAAG-3′

As above-mentioned the invention also relates to a sequence common to LO and HO regions which is localised over the insertion point of the Pervenets mutation. This sequence SEQ ID NO:13 was isolated by RAGE PCR and the sequences of the cloned LO and HO fragments clearly displays the break-up at the insertion point. Primer pairs were defined to amplify across the insertion Pervenets mutation point.

Sequence N2-1F = 5′-TTTTACTCTTTGTTATAATAG-3′
Sequence N3-1R = 5′-TTTTTAGTTCATGGAATCAC-3′
Sequence N2-2F = 5′-ACACTAACACTCATTACATTCG-3′
Sequence N3-2R = 5′-CCTAAAGCTCTGTAGATTTT-3′
Sequence N2-3F = 5′-AAAGCAAAAAACACCGTGATTC-3′
Sequence N3-3R = 5′-GGTGTTATTATTCAGCCTGAA-3′
Sequence N3-3′R = 5′-GATGTTATTATTCAGCCTGAA-3′

The use of these primers respectively leads to
a fragment of 170 bp (SEQ ID NO: 14 and SEQ ID NO:15)
a fragment of 160 bp (SEQ ID NO: 16 and SEQ ID NO:17)
a fragment of 170 bp (SEQ ID NO: 18 and SEQ ID NO:19)
a fragment of 170 bp (SEQ ID NO: 18 and SEQ ID NO:20)

We also demonstrated that sunflower carries also a duplicated sequence at a second locus but where there is no insertion. Thus, the use of these primer pair combinations always revealed these short fragments whatever the LO or HO genotypes. However, this region enables to further characterize revertant mutant that still carry a short insertion that cannot silence the wild desaturase gene, since these were still LO.

Combination of primers amplifying the SSR, the Δ12HOS allele and the sequence across the insertion site will boost improvement of sunflower with high oleic oil content.

Troubles faced by breeders to improved HOAC sunflower are mainly due to the instability of the Pervenets mutation that seems to disappear for two reasons:

1) There is a reversion, corresponding to a deletion in the insertion (several have been characterized) that may lead to the LO phenotype when the silencing mechanism is stopped.

2) There is a suppressor that prevents the silencing mechanism and consequently, the phenotype is LO.

For the reversion the combination of the markers (SSR, Δ12HOS allele and that across the insertion site) enables to unravel what happened. Since there is no genetic recombination between the SSR locus and the Δ12HL locus, the SSR allele will be always linked (in the same phasis) to the Δ12HL allele that was present. If it reverts the Δ12LOR allele will be found linked to the incorrect SSR allele.

Moreover, when the reversion leaves a small DNA fragment in the insertion site, it is possible to directly detect it with the primer pairs described that amplify the sequence over the insertion site.

Changes in the length of the insertion were found in twelve out of 174 recombinant inbred lines. Thus it is frequent and may disturb the expected frequency ratio for HO/LO.

In the progenies, ten individuals carried the SSR of the HO parent (RHA345) linked with a modified insertion. Once corrected the HO LO ratio fits the Mendelian proportion that was distorted since ten LO progenies were in fact revertant.

The suppressor is detected when the presence of the Δ12HOS allele does not lead to the HO phenotype.

### DESCRIPTION OF THE FIGURES

FIG. 1 Δ12HOS and Δ12LOR physical maps established according to EcoRI and/or HindIII RFLP profiles revealed with the Δ12-desaturase cDNA (8).

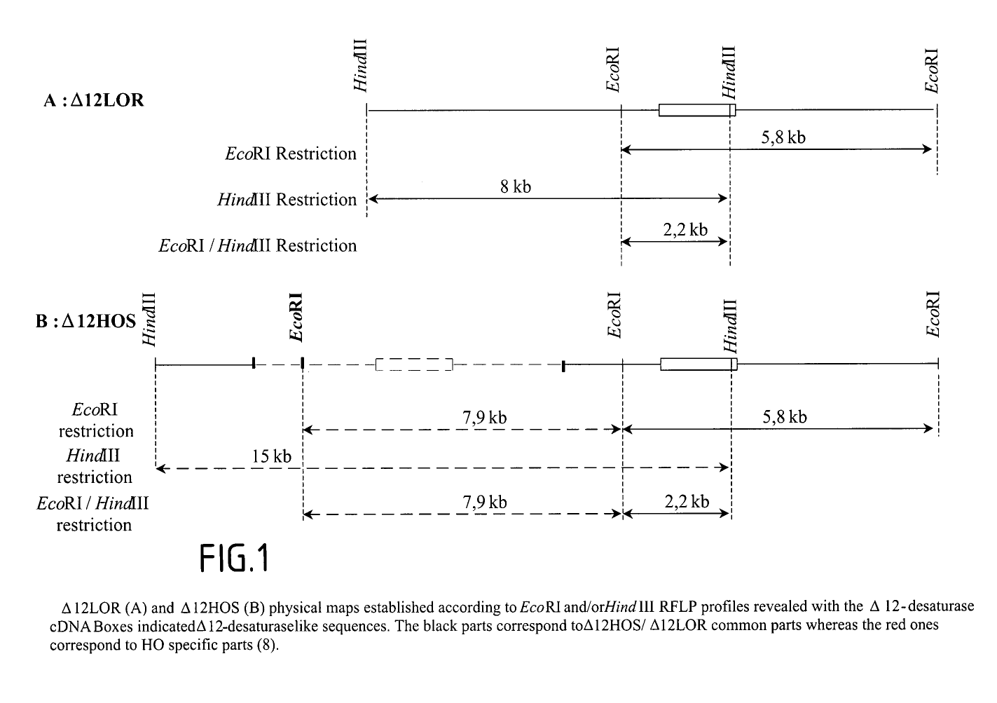

FIG. 2 Outcome map of the 29 kb region with SEQ positions in HO genotypes (B) and in LO genotypes (A).

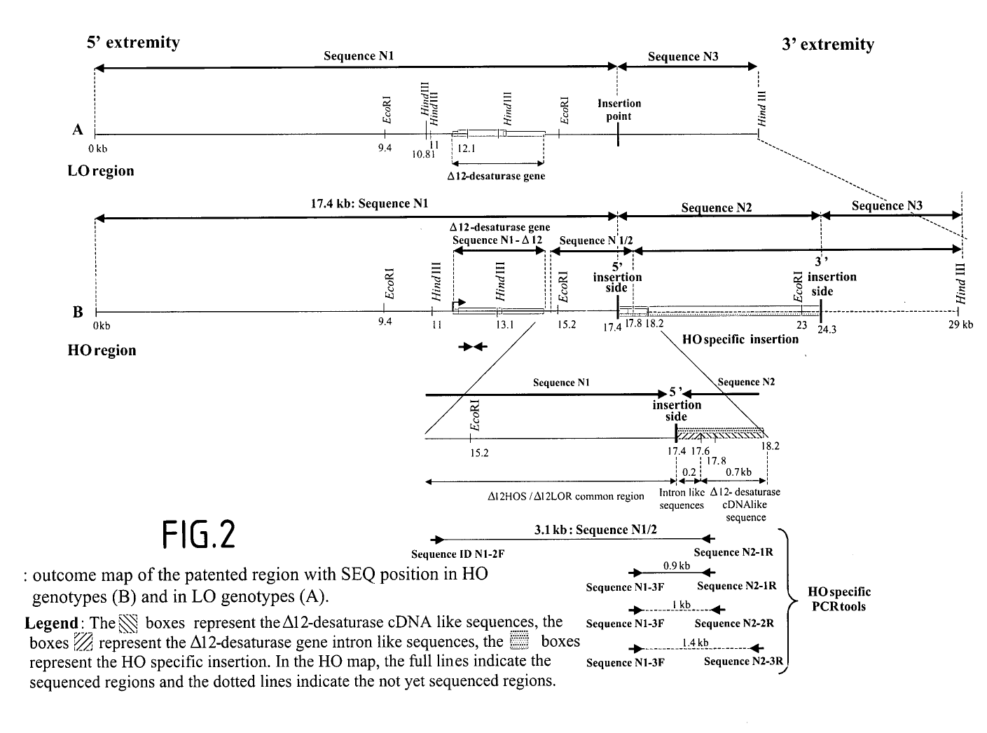

FIG. 3 HO specific PCR amplifications in a set of HO and LO genotypes using primer pair combination SEQ ID N1-3F+SEQ ID N 2-1R (A), SEQ ID N1-3F+SEQ ID N 2-2R (B), SEQ ID N1-3F+SEQ ID N 2-3R (C).

### DESCRIPTION OF MATERIALS AND METHODS

### Materials

Plant Materials

a. Diversity Analysis:

42 HO and LO genotypes to test SSR and other PCR amplification polymorphisms were selected from different public and private institutes in order to represent a wide sunflower genetic diversity. The HO RHA345 and the LO LO4 genotypes (Table 1) were used for long PCR experiments.

TABLE 1
List of genotypes used to test SSR and other PCR amplification
polymorphisms with their pedigree, origin and phenotype.

| | | | PHENOTYPE |
|---|---|---|---|
| CODE | PEDIGREE | ORIGIN | HO/LO |
| LO1 | | Monsanto | LO |
| LO2 | | Monsanto | LO |
| LO3 | | Monsanto | LO |
| LO4 | | Monsanto | LO |
| LO5 | | Monsanto | LO |
| LO11 | | Monsanto | LO |
| LO14 | | Monsanto | LO |
| LO17 | | Monsanto | LO |
| LO36 | | Monsanto | LO |
| LO38 | | Monsanto | LO |
| LO40 | | Monsanto | LO |
| HA89A | VNIIMK8931 | Russia | LO |
| BD70080 | | Monsanto | LO |
| HO1 | | Monsanto | HO |
| HO2 | | Monsanto | HO |
| HO5 | | Monsanto | HO |
| HO9 | | Monsanto | HO |
| HO19 | | Monsanto | HO |
| HO22 | | Monsanto | HO |
| HO24 | | Monsanto | HO |
| HO26 | | Monsanto | HO |
| HO37 | | Monsanto | HO |
| HO39 | | Monsanto | HO |
| HO41 | | Monsanto | HO |
| HO42 | | Monsanto | HO |
| HO43 | | Monsanto | HO |
| OPA1 | PAC2 × RHA344 | INRA - France | HO |
| R-OL1 | | Cordoba | HO |
| OPA2 | PAC2 × RHA344 | INRA - France | HO |
| BE73201-1 | | Monsanto | HO |
| BE-73201-4 | | Monsanto | HO |
| BE-73201-5 | | Monsanto | HO |
| RHA345 | | USA | HO |
| RHA346 | | USA | HO |
| RHA347 | | USA | HO |
| LG26 | | Russia | HO |
| S1 Pervenets | VNIIMK 8931 | Russia | HO |
| S1 Pervenets | VNIIMK 8931 | Russia | HO |
| S1 Pervenets | VNIIMK 8931 | Russia | HO |
| RHA 345 | | USA | HO |
| VNIIMK 8931 | | Russia | LO |

b. Segregating Recombinant Inbred Line Population:

The (LO) line 83HR4 (INRA), male-sterilized by gibberellin, was crossed with the (HO) line RHA345 (USA) in the INRA nursery during the summer of 1996. Nine F₁ hybrid seeds were obtained and the F₁ plants were inter-crossed to produce a F₂ generation in a greenhouse during the following winter. 174 F₆ progenies were obtained from these F₁ plants. These progenies were used to determine 18:1 content separately on half of a cotyledon of five seeds for each F₆ family. These seeds were sown in Jiffypots and after 6 days in a greenhouse they were transferred to the field. For each F₆ family, plant number 2 in the field was selected for molecular characterizations (RFLP and PCR genotyping). 83HR4 and RHA345 parental lines were included as controls.

Probes

Δ12-desaturase cDNA used in the following example has 1458 bp and is similar to the complete Δ12-desaturase cDNA deposited in the GenBank under n^{o} U91341 and described by Hongtrakul et al (5).

Methods

18:1 Oleic Acid Measurement

Oil composition measurement and 18:1 content determination were performed on half a cotyledon using gas chromatography as described by Conte et al. (10).

Extraction of the Genomic DNAs.

The DNA was extracted from 5 g of ground leaves in liquid nitrogen according to the method disclosed by Gentzbittel (11).

After the addition of 9 ml of a sodium sulphate solution (28 mg/ml) prepared in the buffer CTAB 2× (CTAB 2% (w/v), Tris-HCl 10 mM pH8, Na₂ EDTA 100 mM pH8, NaCl 1.4 M, PVP 1% (w/v)), the mixture was incubated at 65° C. for 30 minutes. Five ml of chloroform/isoamylic alcohol (24/1, v/v) was added before centrifugation at 10.000 rpm, 10 min. The supernatant was recovered and incubated at 37° C. for 30 min after the addition of 500 mg RNase A, then for one hour also at 37° C. after the addition of 4 mg of proteinase K. One ml of CTAB 10× (CTAB 10% (w/v), NaCl 0.7 M) and 7 ml of chloroform/isoamylic alcohol (24/1, v/v) was added before centrifugation at 10.000 rpm, 10 min. The supernatant was recovered in a precipitation buffer, the volume of which corresponds to two volumes of the supernatant (CTAB 1% (w/v), Tris-HCl 50 mM pH8, EDTA 100 mM pH8).

After centrifugation at 12.000 rpm, for 15 min., the pellet was recovered and dissolved in 3 ml of TE High buffer (Tris-HCl 10 mM pH8, Na₂ EDTA 1 mM pH8, NaCl 1M) and two volumes of 95% cold ethanol, before a further centrifugation at 12,000 rpm, for 15 min. The pellet containing the DNA was dissolved in 1 ml of TE 0.1× (Tris-HCl 1 mM pH8, Na₂ EDTA 0.1 mM).

The amount of DNA is determined using an absorbance spectrophotometer (DO) at 260 nm.

RFLP Techniques

Southern Blot

Eight μg of DNA of each genotype were restricted with EcoRI and/or HindIII enzymes according to the provider recommendations (Boehringer). Eight enzyme units per μg of DNA were used and each restriction reaction was carried out at 37° C. for 6 hours.

The restriction products were separated by gel electrophoresis on Agarose 0.8% (w/v) gel in the 0.5×TBE buffer (Tris-HCl 45 mM pH8, boric acid 45 mM, EDTA pH8 1 mM) at 1 v/cm for 16 hours. The gel was then colored in EtBr bath (1 μg/ml). The migration profiles were made visible under UV light (at 312 nm).

After 30 min of partial depurination of the DNA by a 0.25 N HCl solution, the samples were transferred onto a Nylon membrane (Appligene). The transfer was made under reduced pressure at 60 mbar during 2 hours using a vacuum transfer apparatus (Appligene). A 0.4 N NaOH solution was used as a transfer solution. The membranes were then washed in a 2×SSC solution (NaCl 3M, sodium citrate 0.3 M pH 7). The DNA was fixed on the membranes by incubation at 80° C. for two hours. The membranes were stocked at 4° C. until using them for hybridization.

Hybridization

The radioactive labelling of the probes was carried out by the random primer elongation in the presence of a desoxyribonucleotide labelled by a radioactive isotype, α³²P-dCTP according to the method described by Feinberg and Volgelstein (12). The prehybridization and the hybridization were carried out in the same buffer containing SDS 7% (w/v), Sodium Phosphate buffer 0.5 M (pH 7.2), EDTA 1 mM (pH 8), stirring at 62° C. for 1 hour (prehybridization) and 12 hours (hybridization). Two washes for 10 min. at 60° C. were carried out using a buffer containing SDS 1% (w/v), Sodium Phosphate buffer 40 mM (pH 7.2), EDTA 1 mM (pH 8) according to methods described by Sambrook et al. (13). The conditions of Na⁺ molarity and of temperature determine stringence conditions high enough to ensure specific hybridizations. An autoradiographic film was then exposed to the membrane at −70° C. for a duration, which depends on the intensity of the radioactive signals emitted by the membrane.

Specific PCR Amplification

Each PCR amplification was carried out starting from 80 ng of genomic DNA in a final volume of 30 μl containing Tris-HCl 10 mM (pH 8.3); KCl 50 mM, MgCl₂ 1.5 mM; dNTPs 200 μM; 1 μM of each primer, Taq polymerase 1U (Sigma). The amplifications were carried out using a PTC 100 thermocycler (MJ Research) according to the following program:

| | |
|---|---|
| denaturation: | 94° C.; 4 min. |
| production (35 cycles): | 94° C.; 1 min. |
| | 55° C.; 1 min. |
| | 72° C.; 1 min. |
| final elongation: | 72° C.; 5 min. |

The amplification products were then either separated by electrophoresis on Agarose gel or purified in order to be cloned and sequenced. The electrophoresis was carried out on 1.2% (w/v) Agarose gel in TBE 0.5× buffer (10 v/cm) for two hours. Purification of the amplification products was carried out with the Wizard PCR Prep DNA Purification Systems kit (Promega).

Amplification of SSR Fragments.

The amplification using the SSR primers was carried out using 40 ng of genomic DNA in a final volume of 25 μl containing Tris-HCl 10 mM (pH 8.3), KCl 50 mM, MgCl₂ 1.5 mM, dNTPs 200 μM, 1 μM of each primer; Taq polymerase 1U (Sigma). The amplifications were carried out using a PTC 100 thermocycler (MJ Research) according to the following program:

| | |
|---|---|
| Denaturation: | 94° C.; 5 min. |
| production (35 cycles): | 94° C.; 30 s. |
| | 50° C.; 1 min. |
| | 72° C.; 1 min. |
| final elongation: | 72° C.; 5 min. |

Amplification product were loaded onto 6% denaturing polyacrylamide gels containing 7.5 M urea, 6% acrylamide and 1×TBE buffer (Tris-HCI 90 mM pH8, boric acid 90 mM, EDTA pH8 2 mM). Gels were run in a 1×TBE buffer for 90 min at 60V. SSR amplifications were visualized by sliver staining with a commercial kit from Promega.

Long PCR Amplification

In order to amplify PCR fragment from genomic DNA longer than 2 kb, we used the Expend Long Template PCR System from Roche Applied Science. The long PCR were made according to the provider instructions.

### EXAMPLE 1

### Diversity Analysis with the HO Specific PCR Fragment Across the Insertion Pervenets Point

The Pervenets mutation was labelled by HO specific PCR amplifications across the 5′ insertion point using designed primer pair combinations (SEQ ID N1-3F located on SEQ ID N1 with SEQ ID N2-1R, -2R or -3R designed on Δ12-desaturase cDNA sequence).

sequence N1-3F = 5′-GAGAAGAGGGAGGTGTGAAG-3′
sequence N2-1R = 5′-AGCGGTTATGGTGAGGTCAG-3′
sequence N2-2R = 5′-ACAAAGCCCACAGTGTCGTC-3′
sequence N2-3R = 5′-GCCATAGCAACACGATAAAG-3′

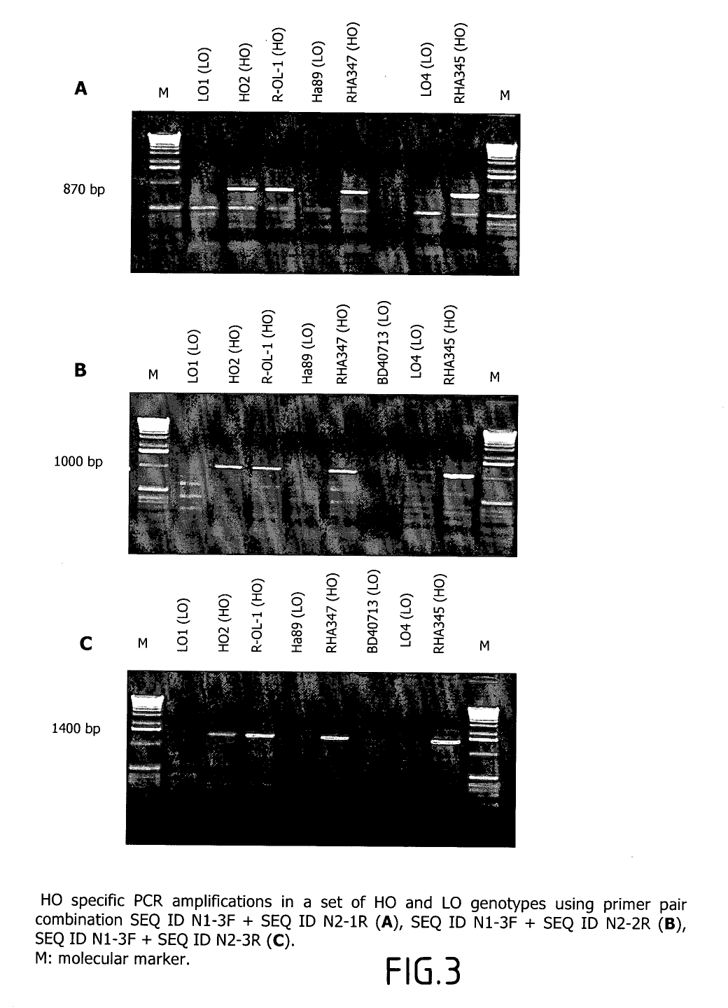

Forty-two HO and LO genotypes were used for these PCR experiments. These genotypes were previously genotyped using Δ12-desaturase cDNA as a probe to reveal RFLPs. All the HO genotypes displayed the Δ12HOS allele whereas the LO genotypes displayed the Δ12LOR one (6). PCR amplification products of about 870 bp (SEQ ID N1-3F+SEQ ID N2-1R), 1000 bp (SEQ ID N1-3F+SEQ ID N2-2R) and 1400 bp (SEQ ID N1-3F+SEQ ID N2-3R) were specifically revealed in HO genotypes carrying the Pervenets mutation (FIG. 3 A, B and C, respectively). All the 42 genotypes were categorized into LO or HO without any error according to these HO specific PCR amplifications.

### EXAMPLE 2

### Pervenets Mutation Labelling in the Recombinant Inbred Line Population

The recombinant inbred line families were obtained by crossing the lines 83HR4 (LO) by RHA 345 (HO) according to the method described above. Using Δ12-desaturase as a probe to reveal RFLP, we showed that the RHA 345 HO parent line carries the Δ12HOS allele whereas the 83HR4 LO parental line displayed the Δ12LOR allele. In the 174 recombinant inbred line population, the Δ12HOS/Δ12LOR alleles segregated as 78/96.

The Pervenets mutation was labelled by the 870 bp PCR fragment across the 5′ insertion point and by the polymorphism of the SSR locus located on the Δ12-desaturase gene intron. Moreover this SSR polymorphism labelled the Δ12-desaturase gene itself.

870 bp HO specific PCR amplification: We used designed primer pair combination SEQ ID N1-3F with SEQ ID N2-1R:

sequence N1-3F = 5′-GAGAAGAGGGAGGTGTGAAG-3′
sequence N2-1R = 5′-AGCGGTTATGGTGAGGTCAG-3′

In the 174 recombinant inbred line population, it leads to PCR amplification of about 870 bp only in genotypes carrying the Δ12HOS allele and thus the Pervenets mutation.

Amplification of the SSR sequence: The SSR polymorphism between RHA 345 and 83HR4 parental line was revealed by PCR amplification using the primer pair SEQ ID N1-1F/SEQ ID N1-1R. This PCR amplification leads to 237 bp (15 SSR motives) and 240 bp (16 SSR motives) in 83HR4 and RHA 345, respectively. The segregation of this SSR polymorphism was tested in the 174 recombinant inbred line population. All the genotypes carrying the Δ12HOS allele displayed the HO RHA 345 SSR polymorphism (16 SSR motives) whereas genotypes carrying the Δ12LOR allele displayed the LO 83HR4 SSR polymorphism (15 SSR motives).

PCR methods with these primer pairs enabling to amplify either the Pervenets mutation itself or the SSR locus, lead to distinguish between genotypes carrying the Pervenets mutation and genotypes without the mutation. Consequently, the SSR sequence and the HO PCR specific fragment may be used in selection programs to identify genotypes carrying the Pervenets mutation. Moreover, the SSR polymorphism can be used to map the Δ12-desaturase gene.

### REFERENCES

(1) Soldatov K I. (1976). Chemical mutagenesis in sunflower breeding. In Proc. VII^{th} Int. Sunflower Conference, Jun. 27-Jul. 3, 1976, Krasnodar.
(2) Garcès R, Garcia J, Mancha M. (1989). Lipid characterization in seeds of a high oleic acid sunflower mutant. Phytochemistry 28 (10): 2597-2600.
(3) Garcés R et Mancha M. (1991). In vitro oleate desaturase in developing sunflower seeds. Phytochemistry 30:2127-2130.
(4) Kabbaj A. et al. (1996). Polymorphism in Helianthus and expression of stearate, oleate and linoleate desaturase genes in sunflower with normal and high oleic content. Helia 19:1-18.
(5) Hongtrakul V, Slabaugh M B, Knapp S J (1998). A seed specific □12 oleate-desaturase gene is duplicated, rearranged and weakly expressed in high oleic acid sunflower lines. Crop Science 38:1245-1249
(6) Lacombe S et Bervillé A (2001). A dominant mutation for high oleic acid content in sunflower (Helianthus annuus L.) seed oil is genetically linked to a single oleate-desaturase RFLP locus. Molecular Breeding 8:129-137.
(7) Lacombe S et al. (2001). An oleate-desaturase and a suppressor locus direct high oleic acid content of sunflower (Helianthus annuus L.) oil in the Pervenets mutant. CR. Acad. Sci., Sciences de la vie 324:839-845.
(8) Lacombe et al. (2002) Genetic, molecular and expression feature of the Pervenets mutant leading to high oleic acid content of seed oil in sunflower. OCL 9:17-23
(9) Devereux, Haeberli and Smithies (1984). A Comprehensive Set of Sequence Analysis Programs for VAX. Nucleic Acids Research 12(1); 387-395
(10) Conte et al. (1989) Half seed analysis: rapic chromatographic determination of the main fatty acids of sunflower seeds. Plant Breeding 102:158-165
(11) Gentzbittel et al. (1994) RFLP studies of genetic relationships among inbred lines of cultivated sunflower (Helianthus annuus L.): evidence of distinct restorer and maintener germplasm pools. Theor. App. Genet 89:419-425.
(12) Feinberg A P et Vogelstein B. (1983). A technic for radiolabelling DNA restriction endonuclease fragments to high specific activity. Analytical Biochemistry 132: 6-13.
(13) Sambrook J, Fritsch E F, Maniatis T. (1989). Molecular cloning: a laboratory manual (2^{nd} edn). Cold Spring Harbor Laboratory Press, Cold Spring Harbor, N.Y.

1. Isolated nucleic acid having the sequence SEQ ID NO: 3.

2. Isolated nucleic acid fragments having 10-30 bp and which hybridize with SEQ ID NO: 2.

3. Isolated nucleic acid fragments according to claim 2, which are selected from
(SEQ ID N^{o}7)
sequence N2-1R: 5′-AGCGGTTATGGTGAGGTCAG-3′
(SEQ ID N^{o}8)
sequence N2-2R: 5′-ACAAAGCCCACAGTGTCGTC-3′
(SEQ ID N^{o}9)
sequence N2-3R: 5′-GCCATAGCAACACGATAAAG-3′

4. Isolated nucleic acid fragments having the sequence SEQ ID NO: 11 or SEQ ID NO: 12.

5. Primer pairs useful for nucleic acid amplification, which comprise:
1) as the first member of the pair a nucleic acid fragment having 10 to 30 bp and which hybridises with sequence N1 (SEQ ID N°1) and
2) as the second member of the pair a nucleic acid fragment having 10 to 30 bp and which hybridises with SEQ ID NO:2.

6. Primers pairs according to claim 5, which comprise as:
1) first member:
(SEQ ID N^{o}5)
sequence N1-2F: 5′-CAAACCAACCACCCACTAAC-3′
or
(SEQ ID N^{o}6)
sequence N1-3F: 5′-GAGAAGAGGGAGGTGTGAAG-3′
2) second member:
(SEQ ID N^{o}7)
sequence N2-1R: 5′-AGCGGTTATGGTGAGGTCAG-3′
(SEQ ID N^{o}8)
sequence N2-2R: 5′-ACAAAGCCCACAGTGTCGTC-3′
(SEQ ID N^{o}9)
sequence N2-3R: 5′-GCCATAGCAACACGATAAAG-3′

7. Isolated nucleic acid having the sequence SEQ ID NO: 13.

8. Isolated nucleic acid fragments having 10-30 bp and which hybridize with SEQ ID NO: 13.

9. Isolated nucleic acid fragments according to claim 8, which are selected from:
(SEQ ID N^{o}14)
sequence N2-1F: 5′-TTTTACTCTTTGTTATAATAG-3′
(SEQ ID N^{o}16)
sequence N2-2F: 5′-ACACTAACACTCATTACATTCG-3′
(SEQ ID N^{o}18)
sequence N2-3F: 5′-AAAGCAAAAAACACCGTGATTC-3′

10. Isolated nucleic acid fragments according to claim 8, which are selected from:
(SEQ ID N^{o}15)
sequence N3-1R: 5′-TTTTTAGTTCATGGAATCAC-3′
(SEQ ID N^{o}17)
sequence N3-2R: 5′-CCTAAAGCTCTGTAGATTTT-3′
(SEQ ID N^{o}19)
sequence N3-3R: 5′-GGTGTTATTATTCAGCCTGAA-3′
(SEQ ID N^{o}20)
sequence N3-3′R: 5′-GATGTTATTATTCAGCCTGAA-3′

11. Process for selecting sunflower having seeds with a high content of oleic acid which comprises the steps of:
extracting the genomic DNA;
amplifying said genomic DNA by means of primer pair, said pair being constituted with a first member of nucleic acid fragment having 10 to 30 bp which hybridises with sequence N1 (SEQ ID NO:1) and a second member of nucleic acid fragment having 10 to 30 bp which hybridises with sequence N2 (SEQ ID NO:2);
hybridizing the amplified DNA fragment with the labelled sequence SEQ ID N° 3 and
isolating the genotypes giving a positive hybridization signal.

12. Process for mapping the Pervenets mutation on the genome, which comprise the steps of:
extracting the genomic DNA;
amplifying said genomic DNA by means of primers SEQ ID N° 11 and SEQ ID N° 12;
isolating the clones having a DNA fragment length which is different from a reference clone without the Pervenets mutation.

13. Process for amplifying the region across the insertion Pervenets mutation site both in LO and HO genotypes, which comprises the steps of:
extracting the genomic DNA;
amplifying said genomic DNA by means of primer pair, said pair being constituted with a first member of nucleic acid fragments having 10 to 30 bpwhich hybridizes with sequence N1 (SEQ ID NO:13).

14. Combination of primers comprising at least a primer pair according to claim 5, at least a primer pair selected from the nucleic acid fragments as defined in claims 8-10 and at least a primer pair having the sequence SEQ ID NO:11 and the sequence SEQ ID NO:12.

15. Test kits for selecting sunflower having seeds with high content of oleic acid comprising at least a primer pair according to claim 5, at least a primer pair selected from the nucleic acid fragments as defined in claims 8-10 and at least a primer pair having the sequence SEQ ID NO:11 and the sequence SEQ ID NO:12.
